(19) United States
(12) Patent Application Publication
(10) Pub. No.: US 2024/0239796 A1
(43) Pub. Date: Jul. 18, 2024

(54) CRYSTALLINE POLYMORPHS OF EPIDERMAL GROWTH FACTOR RECEPTOR INHIBITOR, AND COMPOSITIONS AND METHODS THEREOF

(71) Applicant: Crimson BioPharm Inc., Brookline, MA (US)

(72) Inventors: Jean Zhao, Brookline, MA (US); Xudong Wei, Ningbo (CN); Jane Zheng Li, Kendall Park, NJ (US); Ruiping Wang, Beijing (CN)

(21) Appl. No.: 18/574,248

(22) PCT Filed: Jul. 5, 2022
(86) PCT No.: PCT/US22/36153

§ 371 Date: Dec. 26, 2023

(51) Int. Cl. C07D 471/04; A61K 31/506; C07C 309/29

(52) U.S. Cl. CPC C07D 471/04; A61K 31/506; C07C 309/29; C07B 2200/13

(57) ABSTRACT

Provided herein are crystalline Forms (or polymorphs) I, II, III, IV, V, VI, and VII of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt and methods of inhibiting the activity of EGFR or treating a disease mediated by EGFR by using an effective amount of at least one crystalline form described herein.

### PRIORITY CLAIMS AND RELATED PATENT APPLICATIONS

This application claims the benefit of priority to U.S. Provisional Application Ser. No. 63/144,287, filed Feb. 1, 2021, the entire content of which is incorporated herein by reference.

### TECHNICAL FIELDS OF THE INVENTION

The invention generally relates to pharmaceuticals and therapeutic methods. More particularly, the invention provides crystalline polymorphs of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt and pharmaceutical compositions thereof, as well as methods of their use in treating various diseases and conditions.

### BACKGROUND OF THE INVENTION

The epidermal growth factor receptor (EGFR, ErbB1) belongs to a family of proteins, involved in the proliferation of normal and malignant cells (Artega, C. L., J. Clin. Oncol. 19, 2001, 32-40). Overexpression of Epidermal Growth Factor Receptor (EGFR) is present in at least 70% of human cancers (Seymour, L. K., Curr. Drug Targets 2, 2001, 117-133) such as, non-small cell lung carcinomas (NSCLC), breast cancers, gliomas, squamous cell carcinoma of the head and neck, and prostate cancer (Raymond et al., Drugs 60 Suppl. 1, 2000, discussion 41-2; Salomon et al., Crit. Rev. Oncol. Hematol. 19, 1995, 183-232; Voldborg et al., Ann. Oncol. 8, 1997, 1197-1206). The Epidermal Growth Factor Receptor tyrosine kinase (EGFR-TK) is therefore widely recognized as an attractive target for the design and development of compounds that can specifically bind and inhibit the tyrosine kinase activity and its signal transduction pathway in cancer cells, and thus can serve as either diagnostic or therapeutic agents. For example, the EGFR-TK reversible inhibitor TARCEVA®, is approved by the FDA for treatment of NSCLC and advanced pancreatic cancer. Other anti-EGFR targeted molecules have also been approved including LAPATINIB® and IRESSA®.

Epidermal growth factor receptor (EGFR) tyrosine kinase inhibitors (TKIs) are effective clinical therapies for EGFR mutant advanced non-small cell lung cancer (NSCLC) patients (Mok, T. S., et al., N. Engl. J. Med. 361, 2009, 947-57; Paez, J. G., et al., Science 304, 2004, 1497-500; Lynch, T. J., et al., N. Engl. J. Med. 350, 2004, 2129-39; Rosell, R., et al., Lancet Oncol. 13, 2012, 239-46). Several randomized clinical trials have demonstrated that EGFR TKIs are more effective, as measured by response rate (RR) and progression free survival (PFS), than chemotherapy when used as initial systemic treatment for advanced EGFR mutant NSCLC (Mok, T. S., et al., N. Engl. J. Med. 361, 2009, 947-57; Rosell, R., et al., Lancet Oncol. 13, 2012, 239-46; Sequist, L. V. et al., J. Clin. Oncol. 31, 2013, 3327-34; Wu, Y. L., et al., Lancet Oncol. 15, 2014, 213-22; Maemondo, M., et al. N. Engl. J. Med. 362, 2010, 2380-8; Zhou, C., et al., Lancet Oncol. 12, 2011, 735-42; Mitsudomi, T., et al., Lancet Oncol. 11, 2010, 121-8). However, the vast majority of patients will develop disease progression following successful treatment with an EGFR TKI. The most common mechanism of acquired resistance, detected in 60% of patients, is a secondary mutation in EGFR at position T790 (T790M) (Yu, H. A., et al., Clin. Cancer Res. 19, 2013, 2240-7). This mutation, leads to an increase in ATP affinity, thus making it more difficult for reversible EGFR TKIs gefitinib and erlotinib to bind the EGFR TKI domain (Yun C. H., et al., Proc. Natl. Acad. Sci. USA. 105, 2008, 2070-5).

Covalent EGFR inhibitors have emerged as strategies to inhibit EGFR T790M containing cancers. In pre-clinical models, afatinib, a covalent quinazoline based EGFR inhibitor, is effective both in models harboring only an EGFR activating mutation and in those with a concomitant T790M resistance mutation (Li, D., et al., Oncogene. 27, 2008, 4702-11). However, in lung cancer patients, afatinib is only effective in EGFR TKI naive EGFR mutant cancers and has a RR of <10% in patients with NSCLC that have developed resistance to gefitinib or erlotinib (Miller V. A., et al., Lancet Oncol. 13, 2012, 528-38). Afatinib is a potent inhibitor of both mutant and wild type (WT) EGFR. Inhibition of WT EGFR leads to toxicities, including skin rash and diarrhea, which limits the ability to escalate afatinib doses in patients to those necessary to inhibit EGFR T790M. Irreversible pyrimidine EGFR inhibitors, including the tool compound WZ4002 and clinical compounds CO-1686 and AZD9291, overcome many of the limitations of afatinib (Zhou, W., et al., Nature 462, 2009, 1070-4; Walter, A. O., et al., Cancer Discov. 3, 2013, 1404-15; Cross, D. A., et al., Cancer Discov. 2014). They are not only more potent on EGFR T790M, but also selectively inhibit mutant over WT EGFR and hence should lead to increased clinical efficacy and less toxicity compared with afatinib.

Despite the clinical efficacy of irreversible pyrimidine EGFR inhibitors, it is fully anticipated that patients will ultimately develop acquired resistance to these agents. To date little is known about the mechanisms of acquired resistance and whether cross resistance will occur to all irreversible pyrimidine based and to existing EGFR inhibitors. For these reasons, there remains a need for novel and potent small molecule irreversible pyrimidine EGFR inhibitors.

### SUMMARY OF THE INVENTION

The present invention relates to crystalline Forms (or polymorphs) I, II, III, IV, V, VI, and VII of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt. In certain embodiments, the crystalline forms of the present application have improved stability and suitability for pharmaceutical uses. Other advantages may include favorable pharmacokinetic properties, ease of isolation, process reproducibility, and suitability for large scale manufacturing process.

The present invention also provides pharmaceutical compositions comprising at least one crystalline form described herein and at least one pharmaceutically acceptable excipient.

The present invention additionally provides a method of inhibiting the activity of EGFR in a subject comprising administering to the subject an effective amount of at least one crystalline form described herein or a pharmaceutical composition described herein.

The present invention additionally provides a method of treating a disease mediated by EGFR in a subject comprising administering to the subject an effective amount of at least one crystalline form described herein or a pharmaceutical composition described herein.

The present invention additionally provides use of a crystalline form described herein or a pharmaceutical composition described herein in the manufacture of a medicament for inhibiting the activity of EGFR or treating a disease mediated by EGFR. Also provided is a crystalline form described herein for use in inhibiting the activity of EGFR or treating a disease mediated by EGFR.

Other features or advantages will be apparent from the following detailed description of the drawings and several embodiments, and also from the appended claims.

### BRIEF DESCRIPTION OF THE DRAWINGS

FIG. 1 depicts powder X-ray diffraction (XRPD) patterns of crystalline Forms I, II, III, IV, V, VI, and VII of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

FIG. 2 depicts XRPD patterns of crystalline Form I of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

FIG. 3 depicts photomicrographs of crystalline Form I of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt by Polarizing Microscope (PLM) analysis.

FIG. 4 depicts thermal gravimetric analysis (TGA) and differential scanning calorimetry (DSC) profiles of crystalline Form I of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

FIG. 5 depicts XRPD patterns of crystalline Forms I, II, and II of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt after desolvation and XRPD patterns of crystalline Form III of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt and conversion between Crystalline Forms II and III.

FIG. 6 depicts TGA and DSC profiles of crystalline Form II of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

FIG. 7 depicts TGA and DSC profiles of crystalline Form III of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

FIG. 8 depicts XRPD patterns of crystalline Forms I, III, and IV of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt and conversion between crystalline Forms IV and III.

FIG. 9 depicts TGA and DSC profiles of crystalline Form IV of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

FIG. 10 depicts TGA and DSC profiles of crystalline Form V of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

FIG. 11 depicts TGA and DSC profiles of crystalline Form VI of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

FIG. 12 depicts dynamic vapor sorption (DVS) isotherm plot of crystalline Form VI of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

FIG. 13 depicts TGA and DSC profiles of crystalline Form VII of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

FIG. 14 depicts relationship of crystalline Forms I, II, III, IV, V, VI, and VII of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

FIG. 15 depicts DSC profiles of thermal treatment of crystalline Form I of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

### DETAILED DESCRIPTION OF THE INVENTION

As used herein, the term “compound” refers to N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt. The term “tosylate acid” refers to p-toluenesulfonic acid and the term “tosylate salt” refers to a salt formed between p-toluenesulfonic acid and a basic species.

In some embodiments, the molar ratio of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide to tosylate acid in the compound described herein is 1:1. In some embodiments, the structure of the compound is shown as below:

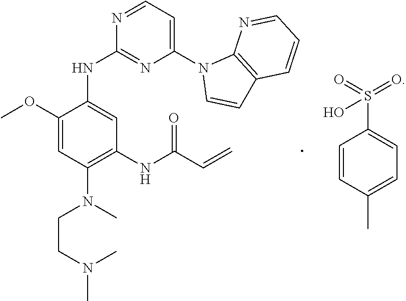 [SMILES: C=CC(=O)Nc1cc(Nc2nccc(-n3ccc4cccnc43)n2)c(OC)cc1N(C)CCN(C)C ; Cc1ccc(S(=O)(=O)O)cc1]

The present invention relates to various crystalline polymorphs of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt and processes for preparing the same. Any suitable crystallization method known in the art can be used to prepare the crystalline forms of the compound described herein. Exemplary crystallization methods include, but are not limited to, anti-solvent precipitation method, slurry conversion crystallization method, evaporation crystallization method, and cooling crystallization method.

As used herein, the term “crystalline”, “crystalline form” or “polymorph” refers to a solid form having a crystal form herein the individual molecules have a highly homogeneous regular locked-in chemical configuration. The crystalline form can be characterized by analytical methods, such as powder X-ray diffraction (XPRD), differential scanning calorimetry (DSC), thermal gravimetric analysis (TGA), dynamic vapor sorption (DVS) analysis, polarizing microscope analysis (PLM), nuclear magnetic resonance (NMR), etc.

As used herein, a “reaction crystallization” method involves reacting the free base of the compound with a chemical agent (e.g., p-toluenesulfonic acid) in a suitable solvent (e.g., MeOH/DCM) at room temperature (RT), followed by adding another solvent (e.g., EtOAc) to form a suspension, and letting the suspension stir at RT for, e.g., 4-7 hours, to precipitate a crystalline form. The precipitate (crystals) may easily be separated by filtration, decanting, or centrifugation.

As used herein, an “anti-solvent precipitation” method involves the addition of an anti-solvent to a solution comprising the compound, which drastically reduces the solubility of the compound and results in the precipitation or crystallization of the compound. The precipitation of the compound can occur immediately or slowly over time. In some embodiments, after the addition of the anti-solvent, the resulting mixture can be maintained at room temperature or cooled to a low temperature (e.g., below room temperature, between 0° C. and 10° C., or between 0° C. and 5° C.) to facilitate the precipitation of the crystalline form. Thereafter, the precipitate (crystals) may easily be separated by filtration, decanting, or centrifugation.

The term “anti-solvent”, as used herein, refers to a solvent in which the compound is insoluble or has very low solubility. Suitable anti-solvents include, but are not limited to, water, hydrocarbons, including petroleum ether, pentane, hexane(s), heptane, octane, isooctane, cyclopentane, cyclohexane, methylcyclohexane, benzene, toluene, xylene, n-butanol.

As used herein “slurry conversion crystallization” method involves stirring of the suspension of the compound in a solvent for a period time sufficient for the conversion of the compound from one solid form to another solid form. In some embodiments, the mixture of the compound and the solvent can be stirred for 1-5 hours, for 1-10 hours, for 1 hour to 1 day, for 1 day to 10 days, or for 1 day to 5 days. In some embodiments, the mixture is stirred for 1 day, 2 days, 3 days, 4 days or 5 days. In some embodiments, the mixture is stirred at room temperature or above room temperature (e.g., 50° C.).

As used herein, “evaporation crystallization” method involves dissolving the compound in several selected solvents to make saturated solutions followed by optional filtration and evaporation at ambient condition and room temperature (e.g., 20° C.) to obtain solids.

As used herein, “cooling crystallization” method involves dissolving the compound in a selected solvent to make a clear solution at a higher temperature (e.g., 50° C. or 60° C.) than room temperature followed by cooling to room temperature (e.g., 20° C.) directly or slowly to obtain solids.

The polymorph screenings or the crystallization methods described herein are conducted using commonly used solvents by various methods including evaporation, slurry, precipitation, cooling crystallization, mechanical and thermal treatment. In some embodiments, the solvents include methanol (MeOH), ethanol (EtOH), isopropyl alcohol (IPA), isobutanol, 2-butanone (MEK), dichloromethane (DCM), tetrahydrofuran (THF), isopropyl acetate (IPAC), acetonitrile (ACN), methyl tert-butyl ether (MTBE), acetone, toluene, water, ethyl acetate (EA), and heptane (HEP).

The term “crystalline Form I”, “crystalline Form II”, “crystalline Form III”, “crystalline Form IV”, “crystalline Form V”, “crystalline Form VI”, or “crystalline Form VII” relates to a specific crystalline form of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

In one aspect, the present invention provides crystalline Form I of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

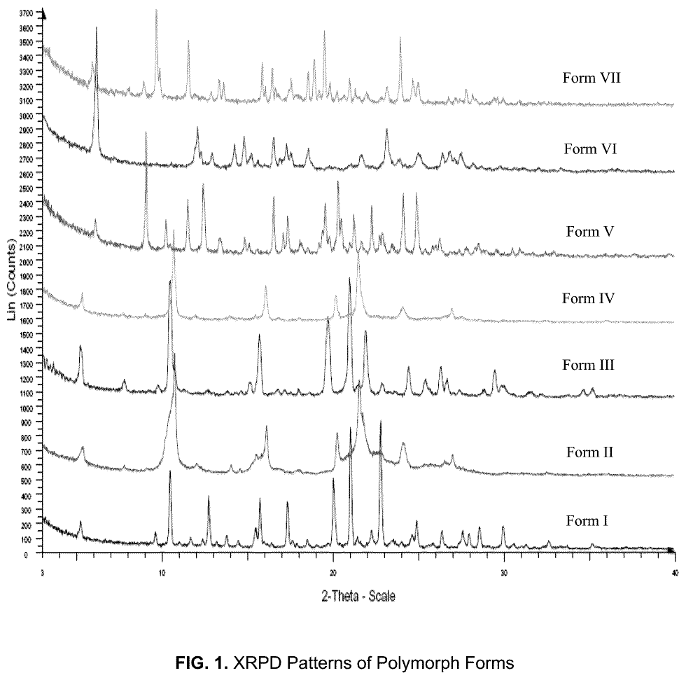

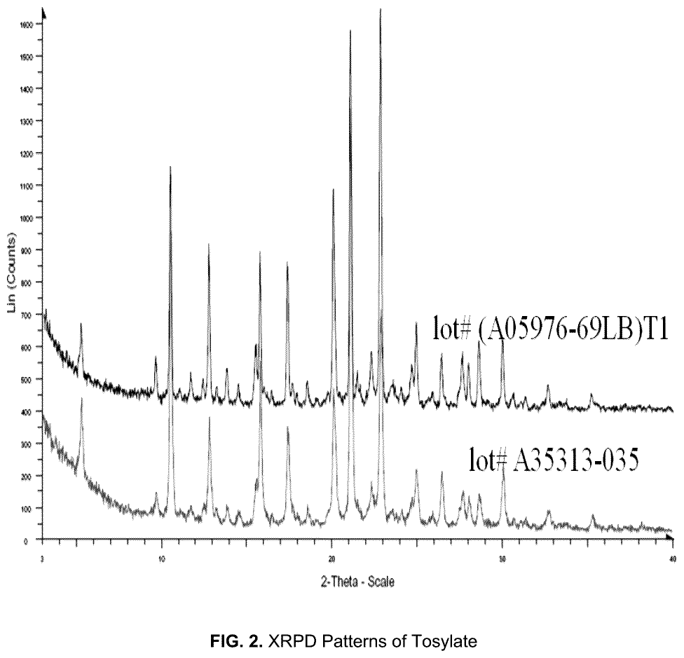

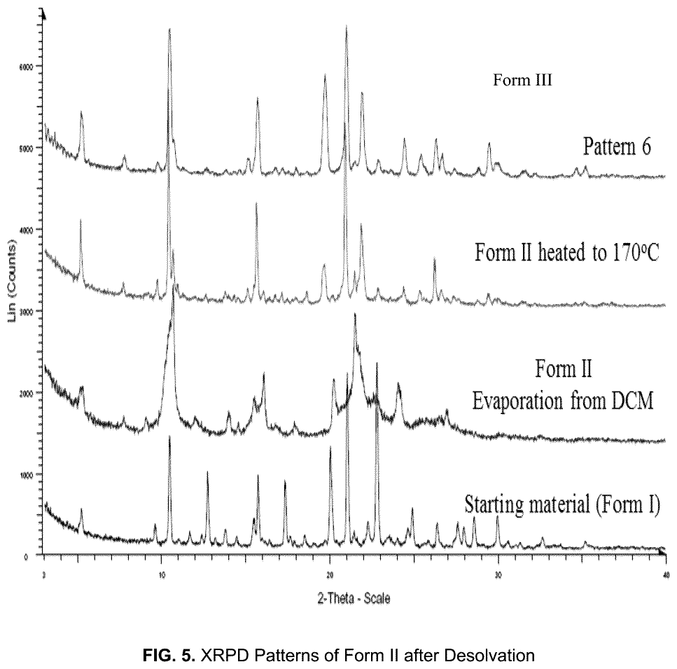

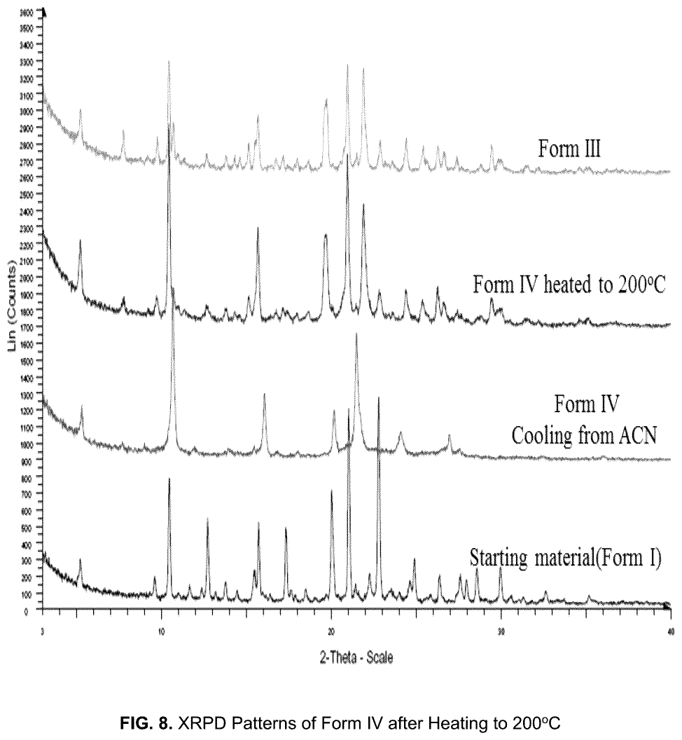

In one embodiment, crystalline Form I is characterized by at least one, at least two, at least three, or at least four X-ray powder diffraction (XRPD) peaks at 2θ angles selected from 10.5°, 17.3°, 20.0°, 21.1°, and 22.8° (0.2°). In another embodiment, crystalline Form I is characterized by XRPD peaks at 2θ angles of 10.5°, 17.3°, 20.0°, 21.1°, and 22.8° (0.2°). In yet another embodiment, crystalline Form I is characterized by at least one, at least two, at least three, at least four, at least five, at least six, or at least seven XRPD peaks at 2θ angles selected from 10.5°, 17.3°, 20.0°, 21.1°, 22.8°, 12.7°, 15.7°, and 24.9° (±0.2°). In some embodiments, the peaks described in the above embodiments for crystalline Form I have a relative intensity of at least 1%, at least 2%, at least 5%, at least 10%, or at least 15%. In some embodiments, the peaks described in the above embodiments for crystalline Form I have a relative intensity of at least 61.5%, at least 36.9%, at least 56%, at least 94.8%, at least 100%, at least 47.3%, at least 42.7%, or at least 100%. In some embodiments, crystalline Form I has a XRPD pattern that is substantially the same as the XRPD pattern designated as “Form I” shown in FIG. 1, or substantially the same as both XRPD patterns shown in FIG. 2, or substantially the same as the XRPD pattern designated as “Starting material (Form I) shown in FIG. 5 or FIG. 8. In some embodiments, crystalline Form I is characterized by XRPD peaks shown in Table 1.

As used herein, the term “relative intensity” refers to a ratio of the peak intensity for the peak of interest versus the peak intensity for the largest peak.

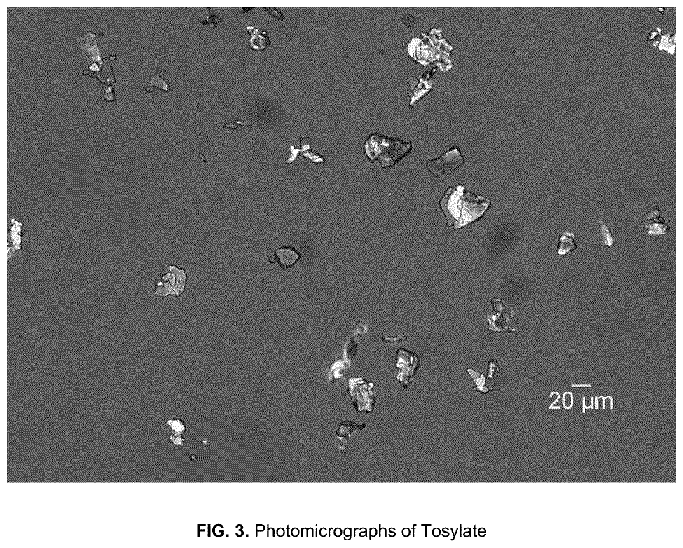

In some embodiments, crystalline Form I has a photomicrograph that is substantially the same as the photomicrograph shown in FIG. 3.

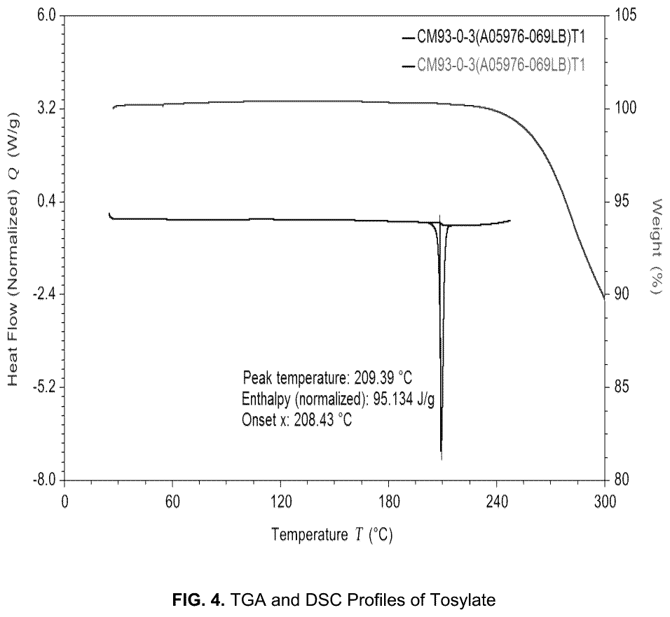

In some embodiments, crystalline Form I has a differential scanning calorimetry (DSC) profile that is substantially the same as DSC profile shown in FIG. 4. In some embodiment, crystalline Form I is characterized by an onset temperature of 208.4° C. in the DSC profile. In another embodiment, crystalline Form I has a melting temperature of 209.4° C. in the DSC profile.

It will be understood that temperatures in DSC profile described herein may vary slightly from one instrument to another and may depend on variation in sample preparation. Therefore, the temperatures are not to be construed as absolute and can vary ±2° C.

In some embodiments, crystalline Form I has a thermal gravimetric analysis (TGA) profile that is substantially the same as the TGA profile shown in FIG. 4. In some embodiment, the TGA profile indicates that crystalline Form I is an anhydrate. As shown in FIG. 4, crystalline Form I has no weight loss in the TGA profile.

In some embodiments, crystalline Form I is characterized by, for example, DSC, TGA and XRPD. In one embodiment, crystalline Form I is characterized by XRPD alone or XRPD in combination with one or more of DSC and TGA described above.

“Anhydrate” or “anhydrous” as used herein, means that the crystalline form comprises substantially no water in the crystal lattice, e.g., less than 1% by weight as determined by, for example, TGA analysis or other quantitative analysis.

In some embodiments, the crystalline Form I is characterized by a purity of about 70% to about 99.9% (e.g., about 80% to about 99.9%, about 90% to about 99.9%, about 95% to about 99.9%).

In some embodiments, crystalline Form I is at least 70%, 80%, 85%, 90%, 95%, 97%, 99%, 99.5% or 99.9% pure. The purity of crystalline Form I is determined by dividing the weight of crystalline Form I in a composition comprising N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt over the total weight of the compound in the composition. In one embodiment, the present invention provides a composition comprising compound N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt, wherein at least 70%, 80%, 85%, 90%, 95%, 97%, 99%, 99.5% or 99.9% by weight of the compound in the composition is crystalline Form I of the compound.

In yet another aspect, the present invention provides a method for preparing crystalline Form I of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt. In one embodiment, the method is reaction crystallization method described herein. In a particular embodiment of the reaction crystallization method, N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide free base was dissolved in MeOH/DCM (1/1) at room temperature (RT), added p-toluenesulfonic acid and then EtOAc to form a suspension, which was stirred at RT for a few hours (e.g., 4 hours, 5 hours, 6 hours, or 7 hours).

In another aspect, the present invention provides crystalline Form II of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

In one embodiment, crystalline Form II is characterized by at least one or at least two XRPD peaks at 2θ angles selected from 10.7°, 21.5°, and 24.10 (0.2°). In another embodiment, crystalline Form II is characterized by XRPD peaks at 2θ angles of 10.7°, 21.5°, and 24.10 (0.2°). In yet another embodiment, crystalline Form II is characterized by at least one, at least two, at least three, at least four, at least five, or at least six XRPD peaks at 2θ angles selected from 10.7°, 21.5°, 24.1°, 10.4°, 21.8°, 16.0°, and 20.3° (0.2°). In some embodiments, the peaks described in the above embodiments for crystalline Form II have a relative intensity of at least 1%, at least 2%, at least 5%, at least 10%, or at least 15%. In some embodiments, the peaks described in the above embodiments for crystalline Form II have a relative intensity of at least 100%, at least 79.1%, at least 50.6%, at least 29%, at least 50.6%, at least 47.3%, at least 42.7%, or at least 37%. In some embodiments, crystalline Form II has a XRPD pattern that is substantially the same as the XRPD pattern designated as “Form II” shown in FIG. 1, or substantially the same as the XRPD pattern designated as “Form II Evaporation from DCM” shown in FIG. 5. In some embodiments, crystalline Form II is characterized by XRPD peaks shown in Table 2.

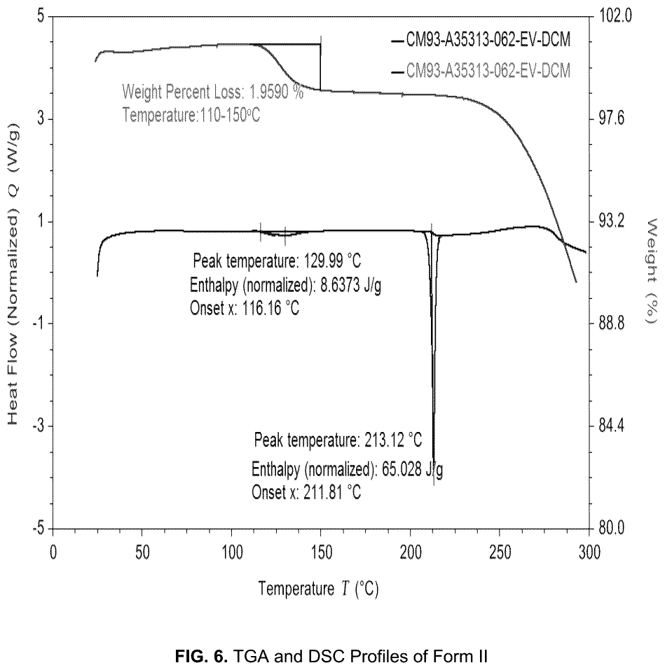

In some embodiments, crystalline Form II has a DSC profile that is substantially the same as DSC profile shown in FIG. 6. In some embodiment, crystalline Form II is characterized by an onset temperature at 116.2° C. in the DSC profile. In some embodiment, crystalline Form II is characterized by an onset temperature at 211.81° C. in the DSC profile. In another embodiment, crystalline Form II has a de-solvation temperature of 130.0° C. In yet another embodiment, crystalline Form II has a melting temperature of 213.1° C.

In some embodiments, crystalline Form II has a TGA profile that is substantially the same as the TGA profile shown in FIG. 6. In particular, the TGA profile indicates that crystalline Form II is a dichloromethane (DCM) anhydrate. In some embodiment, crystalline Form II has 1.9590% weight loss at 110-150° C. in the TGA profile, corresponding to 1.36% residual DCM in NMR.

In some embodiments, crystalline Form II is characterized by, for example, DSC, TGA and XRPD. In one embodiment, crystalline Form II is characterized by XRPD alone or XRPD in combination with one or more of DSC and TGA described above.

As used herein, “solvate” refers to a crystalline solid adduct containing N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt and either stoichiometric or nonstoichiometric amounts of a solvent (e.g., DCM) incorporated within the crystal structure. Techniques known in the art to determine the amount of solvent present include, for example, TGA. In certain embodiments, a solvate is a monosolvate (i.e., one molecule of solvent for every molecule of the compound). In certain embodiments, a solvate is a hemisolvate (i.e., one molecule of solvent for every two molecules of the compound).

In some embodiments, the crystalline Form II is characterized by a purity of about 70% to about 99.9% (e.g., about 80% to about 99.9%, about 90% to about 99.9%, about 95% to about 99.9%).

In some embodiments, crystalline Form II is at least 70%, 80%, 85%, 90%, 95%, 97%, 99%, 99.5% or 99.9% pure. The purity of Form II is determined by dividing the weight of crystalline Form II in a composition comprising N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt over the total weight of the compound in the composition. In one embodiment, the present invention provides a composition comprising compound N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt, wherein at least 70%, 80%, 85%, 90%, 95%, 97%, 99%, 99.5% or 99.9% by weight of the compound in the composition is crystalline Form II of the compound.

In yet another aspect, the present invention provides a method for preparing crystalline Form II of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt. In one embodiment, the method is evaporation crystallization method described herein. In a particular embodiment of the above disclosed method, crystalline Form II can be obtained using crystalline Form I as starting material and DCM as the solvent. In one embodiment, crystalline Form II can be obtained by dissolving crystalline Form I in DCM followed by exposing the solution of crystalline Form I to air at room temperature to allow DCM to evaporate. Alternatively, DCM can be evaporated under vacuum and/or at an elevated temperature (e.g., higher than room temperature). In one embodiment, the concentration of crystalline Form I in DCM is in the range of 1 mg/mL to 10 mg/mL, 2 mg/mL to 9 mg/mL, 3 mg/mL to 7 mg/mL, 4 mg/mL to 6 mg/mL, or 4.5 mg/mL to 5.5 mg/mL. In a particular embodiment, the concentration of crystalline Form I is 5.1 mg/mL.

In yet another aspect, the present invention provides crystalline Form III of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

In one embodiment, crystalline Form III is characterized by at least one or at least two XRPD peaks at 2θ angles selected from 19.5°, 21.7°, and 24.3° (±0.2°). In another embodiment, crystalline Form III is characterized by XRPD peaks at 2θ angles of 19.5°, 21.7°, and 24.3° (±0.2°). In another embodiment, crystalline Form III is characterized by at least one, at least two, at least three, at least four, at least five, or at least six XRPD peaks at 2θ angles selected from 19.7°, 21.7°, 24.3°, 21.0°, 10.4°, 15.7, and 5.2° (±0.2°). In some embodiments, the peaks described in the above embodiments for crystalline Form III have a relative intensity of at least 1%, at least 2%, at least 5%, at least 10%, or at least 15%. In some embodiments, the peaks described in the above embodiments for crystalline Form III have a relative intensity of at least 68.7%, at least 56.8%, at least 27.6%, at least 100%, at least 97.8%, at least 53.8%, or at least 43.8%. In some embodiments, crystalline Form III has a XRPD pattern that is substantially the same as the XRPD pattern designated as “Form III” shown in FIG. 1, or substantially the same as the XRPD pattern designated as “Form III” shown in FIG. 5, or substantially the same as the XRPD pattern designated as “Form III” shown in FIG. 8. In another embodiments, crystalline Form II can transform to crystalline Form III after desolvation at 170° C. as shown in FIG. 5. In some embodiments, crystalline Form III is characterized by XRPD peaks shown in Table 3.

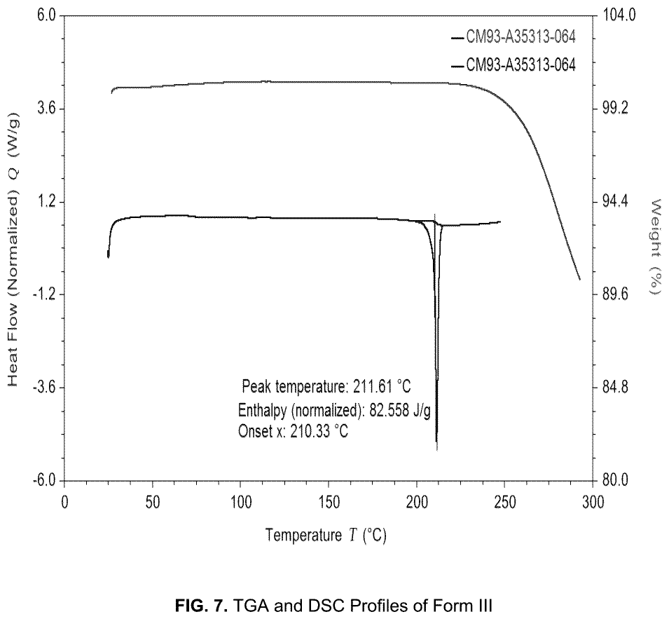

In some embodiments, crystalline Form III has a DSC profile that is substantially the same as DSC profile shown in FIG. 7. In some embodiment, crystalline Form III is characterized by an onset temperature at 210.3° C. in the DSC profile. In another embodiment, crystalline Form III has a melting temperature of 211.6° C.

In some embodiments, crystalline Form III has a TGA profile that is substantially the same as the TGA profile shown in FIG. 7. In particular, the TGA profile indicates that crystalline Form III is an anhydrate. As shown in FIG. 7, crystalline Form III has no weight loss in the TGA profile.

In some embodiments, crystalline Form III is characterized by, for example, DSC, TGA and XRPD. In one embodiment, crystalline Form III is characterized by XRPD alone or XRPD in combination with one or more of DSC and TGA described above.

In some embodiments, the crystalline Form III is characterized by a purity of about 70% to about 99.9% (e.g., about 80% to about 99.9%, about 90% to about 99.9%, about 95% to about 99.9%).

In some embodiments, crystalline Form III is at least 70%, 80%, 85%, 90%, 95%, 97%, 99%, 99.5% or 99.9% pure. The purity of Form III is determined by dividing the weight of crystalline Form III in a composition comprising N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt over the total weight of the compound in the composition. In one embodiment, the present invention provides a composition comprising compound N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt, wherein at least 70%, 80%, 85%, 90%, 95%, 97%, 99%, 99.5% or 99.9% by weight of the compound in the composition is crystalline Form III of the compound.

In yet another aspect, the present invention provides a method for preparing crystalline Form III of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt. In one embodiment, the method is cooling crystallization method described herein. In a particular embodiment of the above disclosed method, crystalline Form III can be obtained using crystalline Form I as starting material and ACN or MeOH-water (19/1) as the solvents by the cooling crystallization method described herein.

In another aspect, the present invention provides crystalline Form IV of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

In one embodiment, crystalline Form IV is characterized by at least one XRPD peak at 20 angles selected from 10.8° and 21.4° (±0.2°). In another embodiment, crystalline Form IV is characterized by XRPD peaks at 2θ angles of 10.8° and 21.4° (±0.2°). In another embodiment, crystalline Form IV is characterized by at least one, at least two, at least three, or at least four XRPD peaks at 2θ angles selected from 10.8°, 21.4°, 16.1°, 5.3°, and 20.2° (±0.2°). In some embodiments, the peaks described in the above embodiments for crystalline Form IV have a relative intensity of at least 1%, at least 2%, at least 5%, at least 10%, or at least 15%. In some embodiments, the peaks described in the above embodiments for crystalline Form IV have a relative intensity of at least 100%, at least 74.9%, at least 40.2%, at least 33.6%, or at least 30.6%. In some embodiments, crystalline Form IV has a XRPD pattern that is substantially the same as the XRPD pattern designated as “Form IV” shown in FIG. 1, or substantially the same as the XRPD pattern designated as “Form IV Cooling From ACN” shown in FIG. 8. In some embodiments, crystalline Form IV is characterized by XRPD peaks shown in Table 4. In some embodiment, crystalline Form IV can convert to crystalline Form III when heated to 200° C. as shown in FIG. 8.

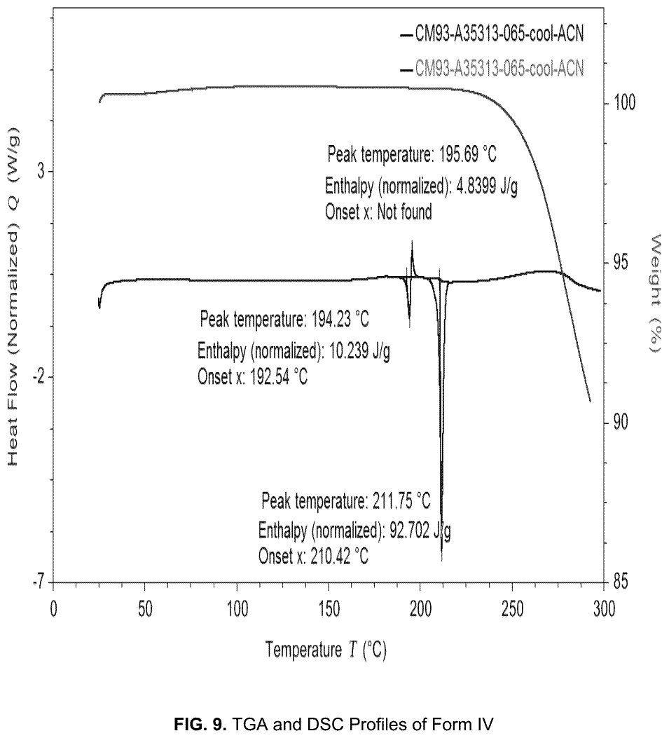

In some embodiments, crystalline Form IV has a DSC profile that is substantially the same as DSC profile shown in FIG. 9. In some embodiment, crystalline Form IV is characterized by an onset temperature at 192.5° C. in the DSC profile. In another embodiment, crystalline Form IV is characterized by an onset temperature at 210.4° C. in the DSC profile. In some embodiment, crystalline Form IV has an endothermic peak of 194.2° C. In another embodiment, crystalline Form IV has a melting temperature of 211.8° C. In some embodiment, crystalline Form IV has an exothermic peak of 195.7° C. as shown in FIG. 9. The exothermic peak indicates that crystalline From IV may first melt and then recrystallize to crystalline Form III as shown in FIG. 8.

In some embodiments, crystalline Form IV has a TGA profile that is substantially the same as the TGA profile shown in FIG. 9. In particular, the TGA profile indicates that crystalline Form IV is an anhydrate. As shown in FIG. 9, crystalline Form IV has no weight loss in the TGA profile.

In some embodiments, crystalline Form IV is characterized by, for example, DSC, TGA and XRPD. In one embodiment, crystalline Form IV is characterized by XRPD alone or XRPD in combination with one or more of DSC and TGA described above.

In some embodiments, the crystalline Form IV is characterized by a purity of about 70% to about 99.9% (e.g., about 80% to about 99.9%, about 90% to about 99.9%, about 95% to about 99.9%).

In some embodiments, crystalline Form IV is at least 70%, 80%, 85%, 90%, 95%, 97%, 99%, 99.5% or 99.9% pure. The purity of Form IV is determined by dividing the weight of crystalline Form IV in a composition comprising N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt over the total weight of the compound in the composition. In one embodiment, the present invention provides a composition comprising compound N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt, wherein at least 70%, 80%, 85%, 90%, 95%, 97%, 99%, 99.5% or 99.9% by weight of the compound in the composition is crystalline Form IV of the compound.

In yet another aspect, the present invention provides a method for preparing crystalline Form IV of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt. In one embodiment, the method is cooling crystallization method described herein. In a particular embodiment of the above disclosed method, crystalline Form IV can be obtained using crystalline Form I as starting material and ACN or MeOH-water as the solvents by the cooling crystallization method described herein. In one embodiment, the ratio of MeOH to water is in the range of 1/5-18/1, 1/3-16/1, 1/2-13/1, 1/1-12/1, 2/1-9/1, or 2/1-7/3. In a particular embodiment, the ratio of MeOH to water is 9/1 or 7/3.

In another aspect, the present invention provides crystalline Form V of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

In one embodiment, crystalline Form V is characterized by at least one, at least two, or at least three XRPD peaks at 2θ angles selected from 9.0°, 12.4°, 20.3° and 22.3° (±0.2°). In another embodiment, crystalline Form V is characterized by XRPD peaks at 2θ angles of 9.0°, 12.4°, 20.3° and 22.3° (±0.2°). In another embodiment, crystalline Form V is characterized by at least one, at least two, at least three, at least four, at least five, at least six, at least seven, at least eight or at least nine XRPD peaks at 2θ angles selected from 9.0°, 12.4°, 20.3°, 22.3°, 24.9°, 24.10, 16.5°, 11.5°, 3.3°, and 19.5° (±0.2°). In some embodiments, the peaks described in the above embodiments for crystalline Form V have a relative intensity of at least 1%, at least 2%, at least 5%, at least 10%, or at least 15%. In some embodiments, the peaks described in the above embodiments for crystalline Form V have a relative intensity of at least 100%, at 60.2%, at least 61.7%, at least 42.3%, at least 53.2%, at least 52.5%, at least 49.6%, at least 47.9%, at least 45.5%, at least 44.4% or at least 42.3%. In some embodiments, crystalline Form V has a XRPD pattern that is substantially the same as the XRPD pattern designated as “Form V” shown in FIG. 1. In some embodiments, crystalline Form V is characterized by XRPD peaks shown in Table 5.

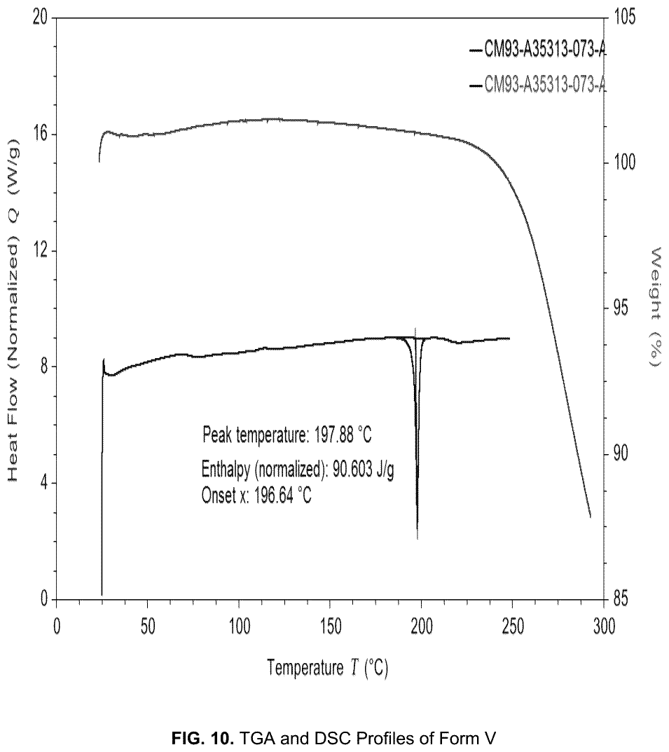

In some embodiments, crystalline Form V has a DSC profile that is substantially the same as DSC profile shown in FIG. 10. In some embodiment, crystalline Form V is characterized by an onset temperature at 196.64° C. in the DSC profile. In another embodiment, crystalline Form V has a melting temperature of 197.88° C. in the DSC profile.

In some embodiments, crystalline Form V has a TGA profile that is substantially the same as the TGA profile shown in FIG. 10. In particular, the TGA profile indicates that crystalline Form V is an anhydrate. As shown in FIG. 10, crystalline Form V has no weight loss in the GA profile.

In some embodiments, crystalline Form V is characterized by, for example, DSC, TGA and XRPD. In one embodiment, crystalline Form V is characterized by XRPD alone or XRPD in combination with one or more of DSC and TGA described above.

In some embodiments, the crystalline Form V is characterized by a purity of about 70% to about 99.9% (e.g., about 80% to about 99.9%, about 90% to about 99.9%, about 95% to about 99.9%).

In some embodiments, crystalline Form V is at least 70%, 80%, 85%, 90%, 95%, 97%, 99%, 99.5% or 99.9% pure. The purity of Form V is determined by dividing the weight of crystalline Form V in a composition comprising N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt over the total weight of the compound in the composition. In one embodiment, the present invention provides a composition comprising compound N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt, wherein at least 70%, 80%, 85%, 90%, 95%, 97%, 99%, 99.5% or 99.9% by weight of the compound in the composition is crystalline Form V of the compound.

In yet another aspect, the present invention provides a method for preparing crystalline Form V of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt. In one embodiment, the method is slurry conversion crystallization method described herein. In a particular embodiment of the above disclosed method, crystalline Form V can be obtained using crystalline Form I as starting material and acetone-water as the solvents by the slurry conversion crystallization method described herein. In one embodiment, the ratio of acetone to water is in the range of 1/6-30/1, 1/3-19/1, 3/7-13/1, 1/2-12/1, 1/1-9/1, or 1/1-7/3. In a particular embodiment, the ratio of acetone to water is 19/1, 9/1, 7/3, 1/1, or 3/7.

In another aspect, the present invention provides crystalline Form VI of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

In one embodiment, crystalline Form VI is characterized by at least one or at least two XRPD peaks at 2θ angles selected from 12.0°, 14.8° and 23.10 (0.2°). In another embodiment, crystalline Form VI is characterized by XRPD peaks at 2θ angles of 12.0°, 14.8° and 23.10 (0.2°). In another embodiment, crystalline Form VI is characterized by at least one, at least two, at least three, at least four, or at least five XRPD peaks at 2θ angles selected from 12.0°, 14.8°, 23.10, 6.10, 16.5°, and 17.3° (±0.2°). In some embodiments, the peaks described in the above embodiments for crystalline Form VI have a relative intensity of at least 1%, at least 2%, at least 5%, at least 10%, or at least 15%. In some embodiments, the peaks described in the above embodiments for crystalline Form VI have a relative intensity of at least 33.1%, at least 26.4%, at least 31.7%, at least 100%, at least 25.6%, or at least 21.7%. In some embodiments, crystalline Form VI has a XRPD pattern that is substantially the same as the XRPD pattern designated as “Form VI” shown in FIG. 1. In some embodiments, crystalline Form VI is characterized by XRPD peaks shown in Table 6.

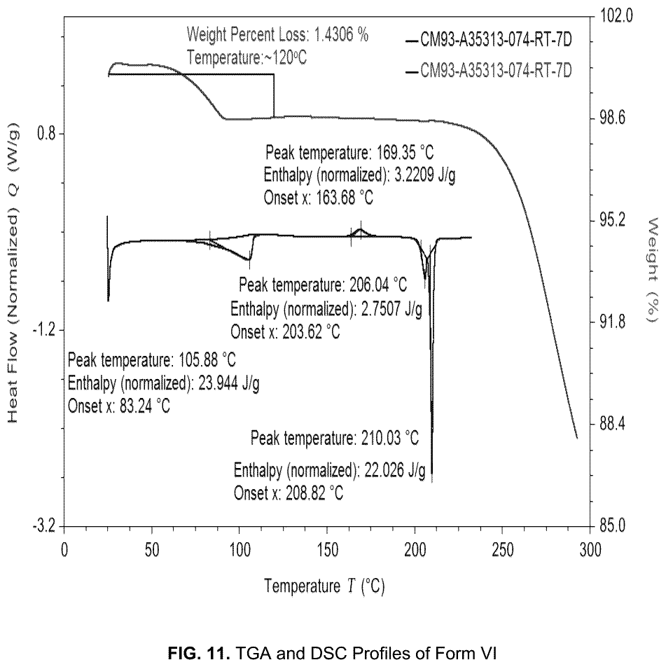

In some embodiments, crystalline Form VI has a DSC profile that is substantially the same as DSC profile shown in FIG. 11. In some embodiment, crystalline Form VI is characterized by an onset temperature of 83.24° C., 203.6° C., and/or 208.8° C. in the DSC profile. In another embodiment, crystalline Form VI has an endothermic peak of 105.9° C., 206.0° C., and/or 210.0° C. in the DSC profile. In some embodiment, crystalline Form VI has an onset temperature of 163.7° C. In another embodiment, crystalline Form VI has an exothermic peak of 169.4° C. The multiple endothermic peaks and the exothermic peak indicate that crystalline From VI may undergo several transformation after dehydration.

In some embodiments, crystalline Form VI has a TGA profile that is substantially the same as the TGA profile shown in FIG. 11. In particular, the TGA profile indicates that crystalline Form VI is a hydrate. As shown in FIG. 11, crystalline Form VI has 1.4% weight loss at 120° C. in the TGA profile, corresponding to water release based on NMR result.

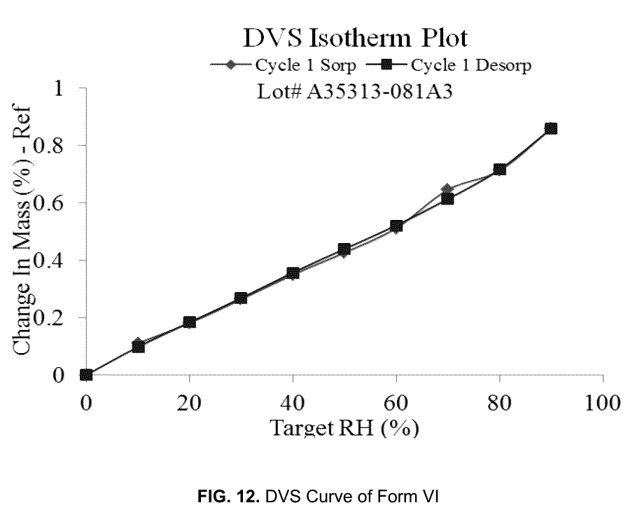

In some embodiments, crystalline Form VI has a dynamic vapor sorption (DVS) isotherm plot substantially the same as the DVS isotherm plot at different target relative humidity (% RH) as shown in FIG. 12. In particular, crystalline Form VI is slightly hygroscopic due to 0.72% water sorption at 80% RH and 25° C. and no crystalline form change is observed after DVS test. In some embodiment, Form VI may be a hydrate.

As used herein, “hygroscopic” as used herein, means that the crystalline form can readily absorb or adsorb water from its surroundings.

As used herein, “hydrate” refers to a crystalline solid adduct containing N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt and either stoichiometric or nonstoichiometric amounts of a water incorporated within the crystal structure. Techniques known in the art to determine the amount of water present include, for example, TGA and Karl Fisher (KF) analysis. In certain embodiments, a hydrate is a monohydrate (i.e., one molecule of water for every molecule of the compound). In certain embodiments, a hydrate is a hemihydrate (i.e., one molecule of water for every two molecules of the compound).

In some embodiments, crystalline Form VI is characterized by, for example, DSC, TGA and XRPD. In one embodiment, crystalline Form VI is characterized by XRPD alone or XRPD in combination with one or more of DSC and TGA described above.

In some embodiments, the crystalline Form VI is characterized by a purity of about 70% to about 99.9% (e.g., about 80% to about 99.9%, about 90% to about 99.9%, about 95% to about 99.9%).

In some embodiments, crystalline Form VI is at least 70%, 80%, 85%, 90%, 95%, 97%, 99%, 99.5% or 99.9% pure. The purity of Form VI is determined by dividing the weight of crystalline Form VI in a composition comprising N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt over the total weight of the compound in the composition. In one embodiment, the present invention provides a composition comprising compound N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt, wherein at least 70%, 80%, 85%, 90%, 95%, 97%, 99%, 99.5% or 99.9% by weight of the compound in the composition is crystalline Form VI of the compound.

In yet another aspect, the present invention provides a method for preparing crystalline Form VI of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt. In one embodiment, the method is slurry conversion crystallization method described herein. In a particular embodiment of the above disclosed method, crystalline Form VI can be obtained using crystalline Form I or a mixture of crystalline Forms I and III as starting material and ACN-water as the solvents by the slurry conversion crystallization method described herein. In one embodiment, the ratio of ACN to water is in the range of 1/20-16/1, 1/12-11/1, or 1/9-7/1. In a particular embodiment, the ratio of ACN to water is 1/9.

In another aspect, the present invention provides crystalline Form VII of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

In one embodiment, crystalline Form VII is characterized by at least one or at least two XRPD peaks at 2θ angles selected from 9.6°, 11.5° and 19.5° (0.2°). In another embodiment, crystalline Form VII is characterized by XRPD peaks at 2θ angles of 9.6°, 11.50 and 19.5° (±0.2°). In another embodiment, crystalline Form VII is characterized by at least one, at least two, at least three, at least four, or at least five XRPD peaks at 2θ angles selected from 9.6°, 11.5°, 19.5°, 23.9°, 3.2°, and 18.9° (±0.2°). In some embodiments, the peaks described in the above embodiments for crystalline Form VII have a relative intensity of at least 1%, at least 2%, at least 5%, at least 10%, or at least 15%. In some embodiments, the peaks described in the above embodiments for crystalline Form VII have a relative intensity of at least 100%, at least 67.6%, at least 77.1%, at least 71%, at least 56.4%, or at least 48.5%. In some embodiments, crystalline Form VII has a XRPD pattern that is substantially the same as the XRPD pattern designated as “Form VII” shown in FIG. 1. In some embodiments, crystalline Form VII is characterized by XRPD peaks shown in Table 7.

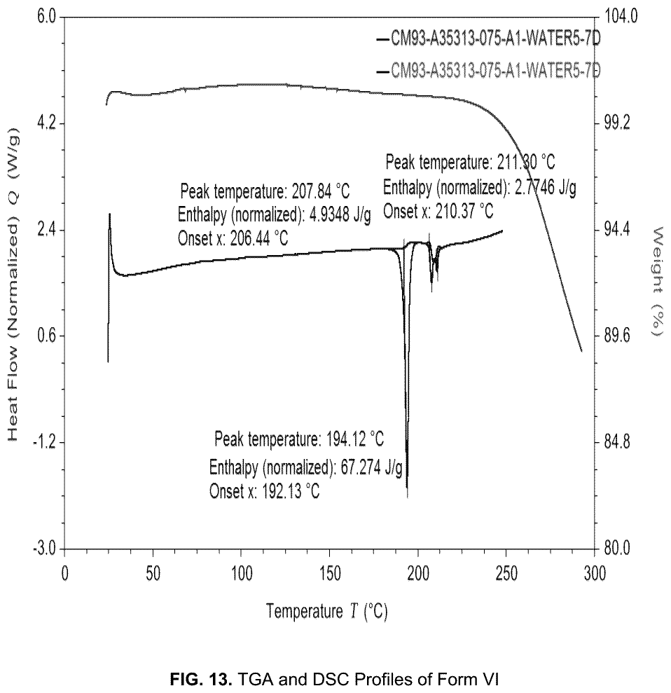

In some embodiments, crystalline Form VII has a DSC profile that is substantially the same as DSC profile shown in FIG. 13. In some embodiment, crystalline Form VII is characterized by an onset temperature of 192.1° C., 206.4° C., and/or 210.4° C. in the DSC profile. In another embodiment, crystalline Form VII has an endothermic peak of 194.1° C., 207.4° C., and/or 211.3° C. in the DSC profile. In some embodiments, crystalline Form VII can recrystallize to other crystalline forms after melting.

In some embodiments, crystalline Form VII has a TGA profile that is substantially the same as the TGA profile shown in FIG. 13. In particular, the TGA profile indicates that crystalline Form VII is an anhydrate. As shown in FIG. 13, crystalline Form VII has no weight loss in the TGA profile.

In some embodiments, crystalline Form VII is characterized by, for example, DSC, TGA and XRPD. In one embodiment, crystalline Form VII is characterized by XRPD alone or XRPD in combination with one or more of DSC and TGA described above.

In some embodiments, the crystalline Form VII is characterized by a purity of about 70% to about 99.9% (e.g., about 80% to about 99.9%, about 90% to about 99.9%, about 95% to about 99.9%).

In some embodiments, crystalline Form VII is at least 70%, 80%, 85%, 90%, 95%, 97%, 99%, 99.5% or 99.9% pure. The purity of Form VII is determined by dividing the weight of crystalline Form VII in a composition comprising N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt over the total weight of the compound in the composition. In one embodiment, the present invention provides a composition comprising compound N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt, wherein at least 70%, 80%, 85%, 90%, 95%, 97%, 99%, 99.5% or 99.9% by weight of the compound in the composition is crystalline Form VII of the compound.

In yet another aspect, the present invention generally relates to a method for preparing crystalline Form VII of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt. In one embodiment, the method is slurry conversion crystallization method described herein. In a particular embodiment of the above disclosed method, crystalline Form VII can be obtained using crystalline Form I as starting material and acetone-water (19/1) as the solvents by the slurry conversion crystallization method described herein.

It will be understood that the 20 values of the XRPD pattern for crystalline Form I, II, III, IV, V, VI, or VII may vary slightly from one instrument to another and may depend on variations in sample preparation. Therefore, the XRPD peak positions for these crystalline Forms are not to be construed as absolute and can vary ±0.2°.

As intended herein, “substantially the same XRPD pattern as shown in FIG. x” mean that for comparison purposes, at least 80%, at least 90%, or at least 95% of the peaks shown in FIG. x are present. FIG. x is FIG. 1, FIG. 2, FIG. 5, or FIG. 8. It is to be further understood that for comparison purposes some variability in peak position from those shown in FIG. x are allowed, such as ±0.2°. Similarly, for comparison purposes some variability in peak position from those shown in the DSC profiles, the TGA profiles, the photomicrographs, and the DVS isotherm plot described herein are allowed. The onset temperature and/or melting temperature and/or exothermic peak temperature can vary from those shown FIG. 4, FIG. 6, FIG. 7, FIG. 9, FIG. 10, FIG. 11, or FIG. 13, such as +2° C.

In yet another aspect, the present invention generally relates to a pharmaceutical composition comprising a crystalline form described herein, e.g., crystalline Form I, II, III, IV, V, VI, or VII, and a pharmaceutically acceptable excipient.

Another aspect of the invention provides a method of inhibiting the activity of EGFR in a subject comprising administering to the subject an effective amount of crystalline Form I, II, III, IV, V, VI, or VII, or a composition (e.g., a pharmaceutical composition) comprising crystalline Form I, II, III, IV, V, VI, or VII.

In one embodiment, crystalline Form I, II, III, IV, V, VI, or VII of the present invention is capable of inhibiting the activity of EGFR containing one or more mutations. In one embodiment, the mutant EGFR contains one or more mutations selected from T790M, L718Q, L844Y, L858R, and Del. In one embodiment, the mutant EGFR contains a combination of mutations, wherein the combination is selected from Del/L718Q, Del/L844Y, Del/T790M, Del/T790M/L718Q, Del/T790M/L844Y, L858R/L718Q, L858R/L844Y, L858R/T790M, and L858R/T790M/L718Q.

In yet another aspect, the invention generally relates to a method of treating a disease mediated by EGFR in a subject comprising administering to the subject an effective amount of crystalline Form I, II, III, IV, V, VI, or VII or a composition (e.g., a pharmaceutical composition) comprising crystalline Form I, II, III, IV, V, VI, or VII.

In some embodiments of the above disclosed aspect, the disease mediated by EGFR is cancer.

In yet another aspect, the invention generally relates to use of
(i) a crystalline Form I of any one of claims 1-7,
(ii) a crystalline Form II of any one of claims 8-14,
(iii) a crystalline Form III of any one of claims 15-21,
(iv) a crystalline Form IV of any one of claims 22-28,
(v) a crystalline Form V of any one of claims 29-35,
(vi) a crystalline Form VI of any one of claims 36-42, and/or
(vii) a crystalline Form VII of any one of claims 43-49,
   for the manufacture of a medicament.

In yet another aspect, the invention generally relates to use of
(i) a crystalline Form I of any one of claims 1-7,
(ii) a crystalline Form II of any one of claims 8-14,
(iii) a crystalline Form III of any one of claims 15-21,
(iv) a crystalline Form IV of any one of claims 22-28,
(v) a crystalline Form V of any one of claims 29-35,
(vi) a crystalline Form VI of any one of claims 36-42, and/or
(vii) a crystalline Form VII of any one of claims 43-49,
   for treating cancer.

The term “cancer” includes diseases or disorders involving abnormal cell growth and/or proliferation, such as glioma, thyroid carcinoma, breast carcinoma, lung cancer (e.g. small-cell lung carcinoma, non-small-cell lung carcinoma), gastric carcinoma, gastrointestinal stromal tumors, pancreatic carcinoma, bile duct carcinoma, ovarian carcinoma, endometrial carcinoma, prostate carcinoma, renal cell carcinoma, lymphoma (e.g., anaplastic large-cell lymphoma), leukemia (e.g. acute myeloid leukemia, T-cell leukemia, chronic lymphocytic leukemia), multiple myeloma, malignant mesothelioma, malignant melanoma, and colon cancer (e.g. microsatellite instability-high colorectal cancer). In some embodiments, the present invention provides a method of treating lung cancer. In some embodiments, the present invention provides a method of treating non-small cell lung cancer (NSCLC). In some embodiments, the present invention provides a method of treating small cell lung cancer (SCLC).

As used herein, the term “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment. In some embodiment, the subject has an EGFR mutation. In another embodiments, the subject has T790M EGFR mutation.

As used herein, the term “treating” or ‘treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; or delaying, inhibiting or decreasing the likelihood of the progression of the disease, disorder or syndrome.

The effective dose of a compound or a crystalline form or a pharmaceutical composition provided herein administered to a subject can be 10 μg-500 mg.

Administering a compound or a crystalline form or a pharmaceutical composition described herein to a mammal comprises any suitable delivery method. Administering a compound or a crystalline form or a pharmaceutical composition described herein to a mammal includes administering a compound or a crystalline form or a pharmaceutical composition described herein topically, enterally, parenterally, transdermally, transmucosally, via inhalation, intracisternally, epidurally, intravaginally, intravenously, intramuscularly, subcutaneously, intradermally or intravitreally to the mammal. Administering a compound or a crystalline form or a pharmaceutical composition described herein to a mammal also includes administering topically, enterally, parenterally, transdermally, transmucosally, via inhalation, intracisternally, epidurally, intravaginally, intravenously, intramuscularly, subcutaneously, intradermally or intravitreally to a mammal a compound or a crystalline form or a pharmaceutical composition that metabolizes within or on a surface of the body of the mammal to a compound or a crystalline form or a pharmaceutical composition described herein.

Thus, a compound or a crystalline form or a pharmaceutical composition described herein, may be systemically administered, e.g., orally, in combination with a pharmaceutically acceptable vehicle such as an inert diluent or an assimilable edible carrier. They may be enclosed in hard- or soft-shell gelatin capsules, may be compressed into tablets, or may be incorporated directly with the food of the patient's diet. For oral therapeutic administration, a compound or a crystalline form or a pharmaceutical composition as described herein may be combined with one or more excipients and used in the form of ingestible tablets, buccal tablets, troches, capsules, elixirs, suspensions, syrups, or wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound or crystalline form in such therapeutically useful compositions can be such that an effective dosage level will be obtained.

The tablets, troches, pills, capsules, and the like can include the following: binders such as gum tragacanth, acacia, corn starch or gelatin; excipients such as dicalcium phosphate; a disintegrating agent such as corn starch, potato starch, alginic acid and the like; a lubricant such as magnesium stearate; or a sweetening agent such as sucrose, fructose, lactose or aspartame or a flavoring agent.

The active compound or crystalline form may also be administered intravenously or intraperitoneally by infusion or injection. Solutions of the active compound or crystalline form can be prepared in water, optionally mixed with a nontoxic surfactant.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

Sterile injectable solutions can be prepared by incorporating the active compound or crystalline form in the required amount in the appropriate solvent with various of the other ingredients enumerated above, as required, followed by filter sterilization. In the case of sterile powders for the preparation of sterile injectable solutions, the preferred methods of preparation can be vacuum drying and the freeze-drying techniques, which can yield a powder of the active ingredient plus any additional desired ingredient present in the previously sterile-filtered solutions.

Exemplary solid carriers can include finely divided solids such as talc, clay, microcrystalline cellulose, silica, alumina and the like. Useful liquid carriers include water, alcohols or glycols or water-alcohol/glycol blends, in which the compound or crystalline form described herein can be dissolved or dispersed at effective levels, optionally with the aid of non-toxic surfactants.

Useful dosages of a compound or a crystalline form described herein can be determined by comparing their in vitro activity, and in vivo activity in animal models. Methods for the extrapolation of effective dosages in mice, and other animals, to humans are known to the art; for example, see U.S. Pat. No. 4,938,949, which is incorporated by reference in its entirety.

The amount of a compound or a crystalline form described herein, required for use in treatment can vary not only with the particular salt selected but also with the route of administration, the nature of the condition being treated and the age and condition of the patient and can be ultimately at the discretion of the attendant physician or clinician. In general, however, a dose can be in the range of from about 0.1 to about 10 mg/kg of body weight per day.

The compound or crystalline form described herein can be conveniently administered in unit dosage form; for example, containing 0.01 to 10 mg, or 0.05 to 1 mg, of active ingredient per unit dosage form. In some embodiments, a dose of 5 mg/kg or less can be suitable.

The desired dose may conveniently be presented in a single dose or as divided doses administered at appropriate intervals.

The disclosed method can include a kit comprising a compound or a crystalline form or a pharmaceutical composition described herein and instructional material which can describe administering a compound or a crystalline form or a composition described herein to a cell or a subject. This should be construed to include other embodiments of kits that are known to those skilled in the art, such as a kit comprising a (such as sterile) solvent for dissolving or suspending a compound or a crystalline form or a composition described herein prior to administering a compound or a crystalline form or a composition described herein to a cell or a subject. In some embodiments, the subject can be a human.

### Examples

### Analytical Methods

### ¹H NMR Analysis

¹H NMR was performed using Bruker Advance 300 equipped with automated sampler (B-ACS 120).

### Powder X-Ray Diffraction (XRPD)

Crystallinity of the compound was examined using D8 advance X-ray diffractometer (Bruker). The system was equipped with LynxEye detector. Samples were scanned from 3 to 40° 2θ, at a step of 0.02° 2θ. The tube voltage and current were 40 KV and 40 mA, respectively.

### Polarizing Microscope Analysis (PLM)

PLM analysis was conducted with a Polarizing Microscope ECLIPSE LV100POL (Nikon, JPN). A sample was put on a piece of glass slide, dispersed with cedar oil and observed with suitable magnification.

### Thermo Gravimetric Analysis (TGA)

TGA was carried out on TGA Q500 or Discovery TGA 55 (TA Instruments, US). A sample was placed in an open tared aluminum pan, automatically weighed, and inserted into the TGA furnace. The sample was heated at 10° C./min to the final temperature.

### Differential Scanning Calorimeter (DSC)

DSC analysis was conducted with DSC Q200 or Discovery DSC 250 (TA Instruments, US). A weighed sample was placed into a DSC pinhole pan, and the weight was accurately recorded. The sample was heated at 10° C./min to the final temperature.

### Dynamic Moisture Sorption Analysis (DVS)

DVS was determined using DVS Intrinsic (SMS, UK). A sample was tested at a targeted relative humidity (RH) of 10 to 90% full cycle in step mode. The analysis was performed in 10% RH increments:
Equilibrium: 60 min;
RH (%) measurement points:
   Sorption: 0, 10, 20, 30, 40, 50, 60, 70, 80, 90
   Desorption: 80, 70, 60, 50, 40, 30, 20, 10, 0.

### Experimental Procedures

### Rough Solubility Testing

About 2 mg of crystalline Form I was weighed into glass vials, and then different selected solvents were added in the vial stepwise until all solid was dissolved. This experiment was conducted by manual dilution combined with visual observation at ˜20° C. The total volume of solvent added was recorded.

### Slurry Study

Appropriate amount of crystalline Form I was added into different solvents to make suspension. The suspensions were kept stirring at room temperature (RT, ˜20° C.) or 50° C., respectively. Solid samples were collected and analyzed by XRPD at a specific time. If new XRPD patterns were identified, the sample was further analyzed by DSC and TGA.

### Anti-Solvent Precipitation

A certain amount of crystalline Form I was dissolved into MeOH to make saturated solution with the concentration of ˜27 mg/mL, and then anti-solvents were added gradually until turbid or 6 mL at RT (˜20° C.). If precipitation occurred, products were characterized accordingly.

### Evaporation Crystallization

The saturated solutions of crystalline Form I in several selected solvents were prepared, filtered and evaporated at ambient condition (˜20° C.). The obtained solids were characterized accordingly.

### Cooling Crystallization

A certain amount (e.g., ˜20 mg) of crystalline Form I was weighed into vials, and the selected solvents were added to make a clear solution at 60° C. Then solutions were cooled to RT (˜20° C.) directly or slowly. Any solids obtained were characterized accordingly.

### Water Activity Study

A certain amount (e.g., ˜70 mg) of crystalline Form I was stirred in 500 μL of acetone-water mixtures with different water content (5%, 10%, 30%, 50%, 70% and 90%) at RT (˜20° C.) for 3, 7 or 8 days. Residual solids were collected and characterized.

### Thermal Treatment

Thermal treatment was conducted using DSC with the parameters below:
Equilibrate at 25° C.,
Ramp 10° C./min to 218° C.,
Ramp 2° C./min to −20° C.,
Ramp 2° C./min to 300° C.

### Stability Study

A certain amount (e.g., ˜10 mg) of crystalline Form I was ground for 2 min or put at 25° C./92.5% RH for 2 weeks. Then the sample was tested by XRPD to determine the physical stability.

### Inter-Conversion Study

The mixture (1:1) of crystalline Form I and crystalline Form III was stirred in IPA at 50° C. (20 mg/mL) and RT (10 mg/mL), and in ACN-water (1/9) at 50° C. (10 mg/mL) and RT (5 mg/mL).

The mixture of crystalline Form I and crystalline Form V of the sample was suspended in IPA at 50° C. (20 mg/mL) and RT (5 mg/mL).

The residual solids from both mixtures were obtained and characterized.

### Results

### 1. Preparation and Characterization of Crystalline Form I

Form I was prepared by reaction recrystallization method. N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide free base was dissolved in 6 mL MeOH/DCM (1/1) at RT and added 111.4 mg of toluenesulfonic acid. Then 24.5 mL of EtOAc was added and the resultant suspension was stirred at RT for 5 hours. The resultant solid was Form I with the major XRPD peaks as listed in Table 1 below.

TABLE 1

| 2θ Angle | Net Intensity | Relative Intensity |
|---|---|---|
| 22.8 | 1274 | 100 |
| 21.1 | 1208 | 94.8 |
| 10.5 | 784 | 61.5 |
| 20.0 | 713 | 56 |
| 12.7 | 543 | 42.6 |
| 15.7 | 519 | 40.7 |
| 17.3 | 470 | 36.9 |
| 24.9 | 299 | 23.5 |

### 2. Preparation and Characterization of Crystalline Form II

From II was prepared by evaporation from DCM using the starting material of Form I. Tosylate Form I (˜20 mg) was dissolved in 4 mL of DCM, and then the filtration was evaporated at ambient conditions. The resultant solid was Form II with the major XRPD peaks as listed in Table 2 below.

TABLE 2

| 2θ Angle | Net Intensity | Relative Intensity |
|---|---|---|
| 10.69 | 1880 | 100 |
| 21.54 | 1487 | 79.1 |
| 10.35 | 952 | 50.6 |
| 21.79 | 889 | 47.3 |
| 16.08 | 803 | 42.7 |
| 20.25 | 696 | 37 |
| 24.13 | 545 | 29 |

### 3. Preparation and Characterization of Crystalline Form III

Form III was prepared in ACN by fast cooling crystallization using Form I as the starting material. Form I (67 mg) was dissolved in 4.2 mL of ACN at 60° C. with stirring. Then the filtration was placed at RT, and the precipitated solid was Form III with the major XRPD peaks as listed in Table 3 below.

TABLE 3

| 2θ Angle | Net Intensity | Relative Intensity |
|---|---|---|
| 21.0 | 1184 | 100 |
| 10.4 | 1158 | 97.8 |
| 19.7 | 813 | 68.7 |
| 21.9 | 673 | 56.8 |
| 15.7 | 637 | 53.8 |
| 5.2 | 519 | 43.8 |
| 24.4 | 327 | 27.6 |

### 4. Preparation and Characterization of Crystalline Form IV

Form IV was prepared in ACN by slow cooling crystallization using Form I as the starting material. Form I (22 mg) was dissolved in 1.4 mL ACN at 60° C. with stirring. Then the solution was cooled slowly to RT. The precipitated solid was Form IV with the major XRPD peaks as shown in Table 4 below.

TABLE 4

| 2θ Angle | Net Intensity | Relative Intensity |
|---|---|---|
| 10.7 | 1049 | 100 |
| 21.5 | 786 | 74.9 |
| 16.1 | 422 | 40.2 |
| 5.3 | 352 | 33.6 |
| 20.2 | 321 | 30.6 |

### 5. Preparation and Characterization of Crystalline Form V

Form V was prepared by slurry in acetone/water at water active 0.72-0.95 at room temperature (RT) for 3 days and 0.31 for 8 days. Form I (50 mg) was stirred in acetone/water (9/1, 7/3, 1/1, or 3/7) at RT for 3 days. Form V was obtained with the major XRPD peaks as shown in Table 5 below.

TABLE 5

| 2θ Angle | Net Intensity | Relative Intensity |
|---|---|---|
| 9.0 | 887 | 100 |
| 20.3 | 547 | 61.7 |
| 12.4 | 534 | 60.2 |
| 24.9 | 472 | 53.2 |
| 24.1 | 466 | 52.5 |
| 16.5 | 440 | 49.6 |
| 11.5 | 425 | 47.9 |
| 3.3 | 404 | 45.5 |
| 19.5 | 394 | 44.4 |
| 22.3 | 375 | 42.3 |

### 6. Preparation and Characterization of Crystalline Form VI

Equal amounts of Form I and II were added into ACN/water (9/1) to make a suspension at 50 mg/mL at room temperature with stirring. The mixed solid was converted to Form VI after stirring at RT for 7 days. The major XRPD peaks of Form VI thus prepared are summarized in Table 6 below.

TABLE 6

| 2θ Angle | Net Intensity | Relative Intensity |
|---|---|---|
| 6.1 | 1506 | 100 |
| 12.0 | 499 | 33.1 |
| 23.2 | 478 | 31.7 |
| 14.8 | 398 | 26.4 |
| 16.5 | 385 | 25.6 |
| 17.3 | 327 | 21.7 |

### 7. Preparation and Characterization of Crystalline Form VII

Form VII was prepared by slurry in acetone/water (19/1, a_{w}=0.31) at room temperature for 8 days. Form I (50 mg) was stirred in acetone/water (19/1) at RT for 8 days. The resultant solid was Form VII with the major XRPD peaks shown in Table 7 below.

TABLE 7

| 2θ Angle | Net Intensity | Relative Intensity |
|---|---|---|
| 9.6 | 528 | 100 |
| 19.5 | 407 | 77.1 |
| 23.9 | 375 | 71 |
| 11.5 | 357 | 67.6 |
| 3.2 | 298 | 56.4 |
| 18.9 | 256 | 48.5 |

### 8. Inter-Conversion Study

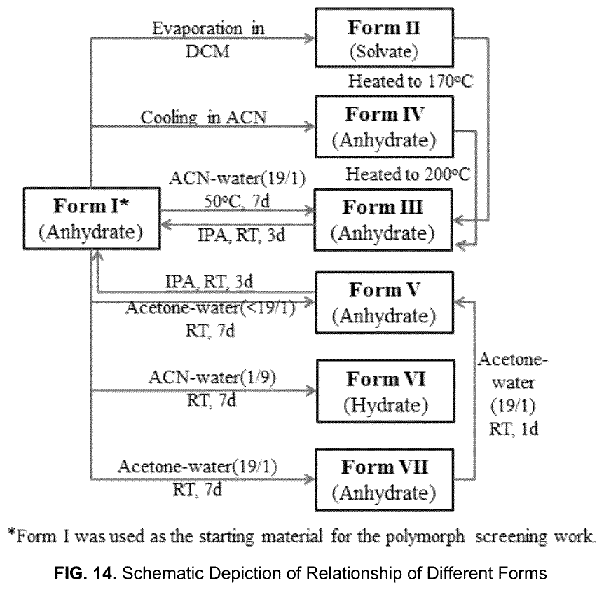

The inter-conversion study result is summarized in FIG. 14. Crystalline Form I had higher melting point and heat fusion as compared to crystalline Form V and crystalline Form VII. As such, crystalline Form I was more stable than them. Crystalline Form I and crystalline Form III might be enantiotopically related. Crystalline Form IV converted to crystalline Form III during heating. Hence, crystalline Forms I and III, both anhydrates, were more stable than other anhydrates. Competitive slurry of crystalline Form I, crystalline Form III and crystalline Form V in IPA showed that all mixtures transformed to crystalline Form I at 50° C. or RT in IPA. However, in slurry experiments, crystalline Form I could transform to crystalline Form V and crystalline Form VII in acetone-water with different water activities. Meanwhile, crystalline Form I also converted to crystalline Form III at 50° C. and to crystalline Form VI at RT in ACN-water (1/9).

Among the anhydrate forms, Form I was considered to be the more stable form. However, presence of water in crystallization media may lead to conversion to the hydrate forms. Preliminary data suggested the solid form conversion was rather complicated in water.

### 9. Polymorph Screening

### 9.1 Preliminary Solubility Study

The solubility of crystalline Form I in 15 single solvents under RT (˜20° C.) was studied, as presented in Table 8. It had relatively high solubility in MeOH and DCM, but very low solubility in other solvents.

TABLE 8

| Solvent | Solubility of Tosylate (mg/mL, ~20° C.) |
|---|---|
| MeOH | ~27.3 |
| EtOH | ~1.90 |
| IPA | <1.11 |
| Isobutanol | <1.0 |
| MEK | ~0.92 |
| THF | <1.14 |
| ACN | ~2.2 |
| MTBE | <1.1 |
| Acetone | ~0.95 |
| Water | <1.02 |
| Toluene | <1.2 |
| EA | <1.0 |
| IPAC | <1.2 |
| DCM | ~5.1 |
| Heptane | <1.0 |

### 9.2 Slurry Study

According to approximate solubility results, 13 different solvents were selected for slurry experiments at RT and 50° C.

### 9.2.1 Slurry at RT (˜20° C.)

The suspension of crystalline Form I (˜25 mg/mL) was stirred in 13 solvents at RT for 4 days. XRPD results (Table 9) illustrated no form change in all slurry samples.

TABLE 9

| Solvent | Form of wet cake at 4 days |
|---|---|
| EtOH | Form I |
| IPA | Form I |
| Isobutanol | Form I |
| MEK | Form I |
| THF | Form I |
| Acetone | Form I |
| MTBE | Form I |
| ACN | Form I |
| Water | Form I |
| Toluene | Form I |
| EA | Form I |
| IPAC | Form I |
| Heptane | Form I |

Note:
starting with Form I

### 9.2.2 Slurry at 50° C.

As shown in Table 10, there was no form change in all slurry samples at 50° C. for 1 day.

TABLE 10

| Solvent | Form of wet cake at 1 day |
|---|---|
| EtOH | Form I |
| IPA | Form I |
| Isobutanol | Form I |
| MEK | Form I |
| THF | Form I |
| Acetone | Form I |
| MTBE | Form I |
| ACN | Form I |
| Water | Form I |
| Toluene | Form I |
| EA | Form I |
| IPAC | Form I |
| Heptane | Form I |

Note:
starting with Form I

### 9.3 Anti-Solvent Precipitation Method

According to solubility test results, MeOH was selected as a solvent for anti-solvent crystallization using crystalline Form I as starting material. As shown in Table 11, solids came out from EA, IPA and MTBE, but other samples were clear solutions. All solids did not undergo form change.

TABLE 11

| | |
|---|---|
| Anti-solvent | Form of wet cake |

| | | |
|---|---|---|
| IPA | 4.9 mL | Form I |
| MTBE | 3.4 mL | Form I |
| THF | 6.0 mL | Clear |
| Acetone | 6.0 mL | Clear |
| Water | 6.0 mL | Clear |
| Toluene | 6.0 mL | Clear |
| EA | 6.0 mL | Form I |

Note:
Room Temp, starting with Form I

### 9.4 Evaporation Crystallization

Evaporation experiments were conducted in MeOH and DCM. Crystalline Form II was obtained in DCM. The mixture of crystalline Forms I and III were obtained in MeOH. The results are shown in Table 12.

TABLE 12

| Solvent | Concentration (mg/mL) | Results |
|---|---|---|
| DCM | ~5.1 | Form II |
| MeOH | ~10.0 | Mixture of Form I and III |

Note:
RT, Form I as staring material

### 9.5 Cooling Crystallization

As crystalline Form I had low solubility in most of organic solvents, cooling crystallization was performed in the selected solvents including EtOH, ACN and mixture of MeOH-water, as shown in Table 13 and Table 14. Pure crystalline Form III and crystalline Form IV was found in ACN and MeOH-water, but sometimes the mixture of crystalline Forms I and III would appear in ACN. Different forms were obtained from MeOH-water mixture with different water content and different cooling rate resulted in different forms in ACN.

TABLE 13

| Solvent | Cooling (60° C. to RT) | Form of wet cake |
|---|---|---|
| EtOH | Crash cooling | Form I |
| ACN | Crash cooling | Mixture of Form I and III |
| | Crash cooling | Form III |
| | Crash cooling | Form III |
| | Slow cooling | Form IV |

Note:
starting with Form I

TABLE 14

| MeOH/water (v/v) | Concentration(mg/mL) | Form |
|---|---|---|
| 19/1 | 86.1 | Form III |
| 9/1 | 136.4 | Form IV |
| 7/3 | 90.7 | Form IV |
| 1/1 | 54.6 | Form I |

Note:
starting with Form I

### 9.6 Water Activity Study

To study if new polymorph or hydrate would be obtained under conditions of different water activities, slurry experiments using crystalline Form I as starting material in acetone-water mixtures with different ratio were carried out at RT. As shown in Table 15, crystalline Form V and crystalline Form VII were obtained. There were different final forms with different water activities for 7 days or 8 days. Crystalline Form I firstly transformed to crystalline Form VII, then to crystalline Form V in acetone-water (19/1).

TABLE 15

| Acetone/water | Water | | | |
|---|---|---|---|---|
| (v/v) | activity | Form at 3 d | Form at 7 d | Form at 8 d |
| 19/1 | 0.31 | Form I | Form VII | Form V |
| 9/1 | 0.72 | Form V | Form V | / |
| 7/3 | 0.88 | Form V | Form V | / |
| 1/1 | 0.93 | Form V | Form V | / |
| 3/7 | 0.95 | Form V | Form V | / |
| 1/9 | 0.98 | Form I | Form I | / |

Note:
RT, starting with Form I

### 9.7 Thermal Treatment

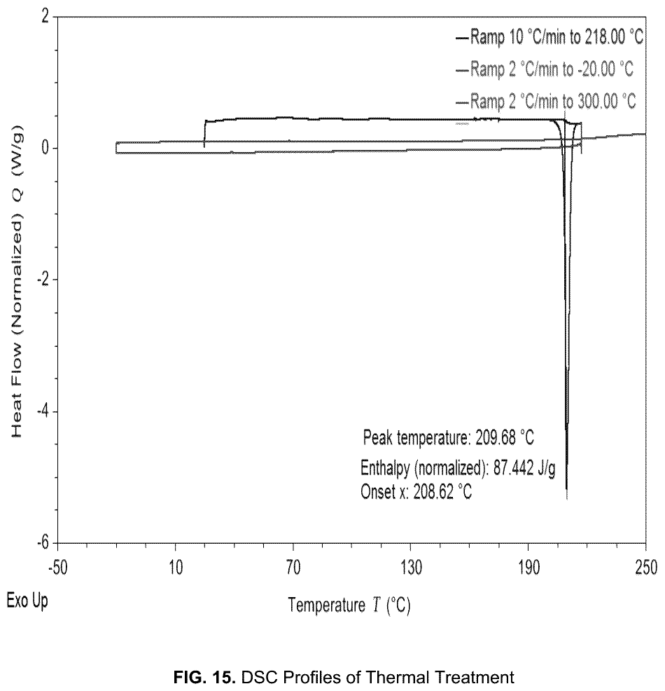

Thermal treatment using crystalline Form I as staring material was employed to obtain possible crystal forms. As shown in FIG. 15, an endothermic peak with onset temperature of 208.62° C. was detected, corresponding to the melting of crystalline Form I. Glass transition (Tg ˜95° C.) was observed. No new form was found.

### 9.8 Stability Study

Physical stability of crystalline Form I was evaluated under mechanical stress (grinding) and high humidity (25° C./92.5% RH for 2 weeks). No form change was observed. Hence Form I was physically stable under grinding and high humidity.

### 9.9 Inter-Conversion Study

Competitive slurry of crystalline Form I, crystalline Form III and crystalline Form V in IPA showed that all mixtures transformed to Form I at 50° C. and RT (Table 16).

TABLE 16

| | | Slurry at RT (~20° | Slurry at 50° C. |
|---|---|---|---|
| Solvent | Starting Material | C.) 3 d (wet cake) | 1 or 7 d (wet cake) |
| IPA | Form I and III (1:1) | Form I | Form I |
| IPA | Form I and V (1:1) | Form I | From I |

When the mixture of crystalline Forms I and III was stirred at different temperatures in ACN-water, different forms were obtained as crystalline Form VI at RT and crystalline Form III at 50° C. (Table 17). The final form might depend on solvents in some degree.

TABLE 17

| Temperature | ACN/water (v/v) | Form at 7 d |
|---|---|---|
| RT (~20° C.) | 1/9 | Form VI |
| 50° C. | 1/9 | Form III |

Note:
starting with the mixture of Form I and Form III

Polymorph screening of crystalline Form I of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt was conducted to search new crystal forms by various methods containing slurry, evaporation, anti-solvent precipitation, cooling crystallization, and water activity study, mechanical and thermal treatment. Seven forms were identified including a DCM solvate (crystalline Form II), a hydrate (crystalline Form VI), and five anhydrates (crystalline Forms I, III, IV, V and VII), and the starting material was crystalline Form I.

The results from this polymorph screening indicated that crystalline Form I was the most stable anhydrate form. Crystalline Form I also showed good solid-state stability after stressing with temperature and high RH. In addition, crystalline Form I was easy to prepare and reproduce.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. Crystalline Form I of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

2. The crystalline Form I of claim 1, wherein the crystalline Form I is characterized by at least one, at least two, at least three, or at least four X-ray powder diffraction (XRPD) peaks at 2θ angles selected from 10.5°, 17.3°, 20.0°, 21.1°, and 22.8° (±0.20).

3. The crystalline Form I of claim 1, wherein the crystalline Form I is characterized by XRPD peaks at 2θ angles of 10.5°, 17.3°, 20.0°, 21.1°, and 22.8° (0.20).

4. The crystalline Form I of claim 1, wherein the crystalline Form I is characterized by a melting temperature of about 209.4° C. determined by differential scanning calorimetry (DSC) analysis.

5. The crystalline Form I of claim 1, wherein the crystalline Form I is an anhydrate.

6. The crystalline Form I of claim 1, wherein the crystalline Form I is characterized by a purity of about 70% to about 99.9%.

7. The crystalline Form I of claim 1, wherein the crystalline Form I is at least 70%, 80%, 85%, 90%, 95%, 97%, 99%, 99.5%, or 99.9% pure.

8. Crystalline Form II of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

9. The crystalline Form II of claim 8, wherein the crystalline Form II is characterized by at least one or at least two XRPD peaks at 2θ angles selected from 10.7°, 21.5°, and 24.1° (±0.2°).

10. The crystalline Form II of claim 8, wherein the crystalline Form II is characterized by XRPD peaks at 2θ angles of 10.7°, 21.5°, and 24.1° (±0.2°).

11. The crystalline Form II of claim 8, wherein the crystalline Form II is characterized by a melting temperature of about 213.1° C. after desolvation determined by DSC analysis.

12. The crystalline Form II of claim 8, wherein the crystalline Form II is a dichloromethane solvate.

13. The crystalline Form II of claim 8, wherein the crystalline Form II is characterized by a purity of about 70% to about 99.9%.

14. The crystalline Form II of claim 8, wherein the crystalline Form II is at least 70%, 80%, 85%, 90%, 95%, 97%, 99%, 99.5%, or 99.9% pure.

15. Crystalline Form III of N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide tosylate salt.

16. The crystalline Form III of claim 15, wherein the crystalline Form III is characterized by at least one or at least two XRPD peaks at 2θ angles selected from 19.5°, 21.7°, and 24.3° (±0.2°).

17. The crystalline Form III of claim 15, wherein the crystalline Form III is characterized by XRPD peaks at 2θ angles of 19.5°, 21.7°, and 24.3° (±0.2°).

18. The crystalline Form III of claim 15, wherein the crystalline Form III is characterized by a melting temperature of about 211.6° C. determined by DSC analysis.

19. The crystalline Form III of claim 15, wherein the crystalline Form III is an anhydrate.

20-49. (canceled)

50. A pharmaceutically composition comprising a pharmaceutically acceptable excipient and
a crystalline Form I of claim 1.

51-65. (canceled)
